(19) United States
(12) Patent Application Publication
(10) Pub. No.: US 2025/0312493 A1
(43) Pub. Date: Oct. 9, 2025

(54) FLUOROPHORE-LOADED GELATIN-BASED NANOPARTICLES FOR NEAR-INFRARED IMAGING

(71) Applicant: The Board of Trustees of the University of Illinois, Urbana, IL (US)

(72) Inventors: Hyungsoo CHOI, Champaign, IL (US); Kyekyoon KIM, Champaign, IL (US); Benjamin LEW, Champaign, IL (US); Viktor GRUEV, Champaign, IL (US)

(73) Assignee: The Board of Trustees of the University of Illinois, Urbana, IL (US)

(21) Appl. No.: 18/866,044

(22) PCT Filed: May 30, 2023
(86) PCT No.: PCT/US2023/023841

§ 371 Date: Nov. 14, 2024

Related U.S. Application Data
(60) Provisional application No. 63/347,406, filed on May 31, 2022.

(51) Int. Cl. A61K 49/00

(52) U.S. Cl. CPC A61K 49/0073; A61K 49/0034; A61K 49/0056

(57) ABSTRACT

Provided are fluorescent nanoparticle and related methods of using the fluorescent nanoparticles for imaging tumors. The fluorescent nanoparticle comprises a gelatin matrix; and a fluorophore encapsulated within the gelatin matrix and ionically-bound to the gelatin matrix. The encapsulated fluorophore has an encapsulation concentration selected to provide: quenching of fluorophore encapsulated within the gelatin matrix and a substantial fluorescence for the fluorophore released from the gelatin matrix.

### CROSS-REFERENCE TO RELATED APPLICATIONS

This application claims the benefit of priority to U.S. Provisional Patent Application No. 63/347,406, filed May 31, 2022, which is hereby incorporated by reference in its entirety to the extent not inconsistent herewith.

### BACKGROUND

Provided herein are fluorescent nanoparticles formed from gelatin that encapsulates a fluorophore that are particularly useful for biological applications where near-infrared (NIR) imaging of the fluorophore is desirable.

Identifying and differentiating malignant tumor from normal tissue during intraoperative procedures is crucial for complete tumor resection. To address this, imaging techniques, such as optical (fluorescence and Raman), acoustic (photoacoustic and radiofrequency), and nuclear imaging-based approaches, are being investigated. Among these approaches, fluorescence imaging, highly compatible with the intraoperative setting in the near-infrared (NIR) window, emerges as a promising modality due to the low tissue absorption, low tissue scattering and low auto-fluorescence of the NIR light, allowing deep tissue imaging to provide surgeons with real-time visual guidance for tumor resection when used with corresponding exogenous fluorophores. Many NIR fluorophores, however, have challenges associated with in vivo applications, including for tumor imaging. For example, indocyanine green (ICG) is an NIR fluorophore approved by the Food and Drug Administration for clinical use. However, ICG exhibits photo- and thermal-instability in aqueous solutions, high protein binding, and short circulation half-life. These drawbacks limit the amount of ICG delivered to tumors, lowering the contrast enhancement and restricting its practical applications. These challenges are not confined to ICG, but are broadly problematic for fluorophores in general, including NIR fluorophores.

Other imaging techniques suffer from various disadvantages. As one example, use of carriers for imaging, such as gold nanorods, mesoporous silica, graphene oxide, chitosan nanoparticles, and liposomes have been employed. However, they exhibit low biodegradability, leading to long-term accumulation-induced toxicity and safety risks for in vivo applications. Liposomes can encapsulate and deliver a wide range of drugs with controllability in degradation, targeting, and toxicity; yet, they have low encapsulation efficiency and poor storage stability when encapsulating water-soluble drugs leading to rapid leakage into the systemic circulation. Chitosan, a natural polymer, could be formulated as cationic nano-complexes with dye. But, it exhibits low water solubility, charge-dependent cytotoxicity and resistivity against enzymatic degradation. Poly(lactic-co-glycolic acid) (PLGA) nanoparticles are hydrophobic, requiring chemical modifications with water-soluble moieties to incorporate water-soluble ICG.

The fluorescent nanoparticles described herein address the problems in the art summarized above by encapsulating a fluorophore in a gelatin matrix such that there is minimal or no fluorescence emission for the encapsulated fluorophores and optically-detectable fluorescence for fluorophores that are released from the gelatin matrix by protease activity in the tumor environment.

### SUMMARY

The instant fluorescent nanoparticles address the problems associated with maintaining fluorophore functionality and selective imaging of tumors by providing fluorophore-loaded gelatin-based nanoparticles, which emit no or minimal fluorescence until degraded by proteases secreted by tumors. Collagen is one of the major components of the tumor microenvironment that regulates tumor infiltration, angiogenesis, and migration. Gelatin, derived from collagen via either acid or base hydrolysis, is generally recognized as safe (GRAS) by the FDA. Excellent compatibility of gelatin with the collagen matrix promotes the interaction between the instant fluorescent nanoparticles and the tumor microenvironment to enhance cellular uptake of the fluorescent nanoparticles in and around tumors. The overexpression of matrix metalloproteinases (MMPs) and proteases in the tumor microenvironment facilitates the degradation of the internalized particles to release ICG intratumorally to achieve improved contrast for image-guided surgery. In contrast, the fluorescent nanoparticles that are not in the tumor microenvironment tend to contain the fluorophores within the gelatin matrix where the fluorophores are quenched in a manner that there is not any significant optically detectable fluorescence.

The common aspect of the fluorescent nanoparticles described herein is the stronger contrast enhancement of the fluorophore by its encapsulation in gelatin-based nanoparticles, which protect the fluorophore from the physiological environment to increase circulation time and facilitate its delivery to and accumulation in tumors. Encapsulated the fluorophore molecules are bound to the gelatin matrix via ionic interactions without covalent conjugation. The fluorescence emission of the encapsulated and bound fluorophore is quenched until the fluorescent nanoparticles are delivered to tumor tissues through the leaky tumor vasculature. For example, there is total quenching at fluorophore concentrations as low as 10 μM ICG. This means that no measurable fluorescence signal is detected until the matrix is degraded by the proteases secreted by tumor cells. Upon degradation of the gelatin matrix by intratumoral proteases and proteinases, the fluorophore is released and its emission regained, resulting in improved fluorescence tumor-to-background ratios (TBR). Free (unbound) ICG fluorophore has strong fluorescent signals without quenching for concentrations in the about 50 μM to 200 μM range. In addition, the fluorescent nanoparticles provided herein enable efficacious intratumoral fluorophore (ICG) delivery, achieving high TBRs as early as 2 hours after its administration with a dose (1.0 mg/kg ICG) significantly lower than the conventional dose (5-10 mg/kg ICG) without encapsulating gelatin matrix.

Provided herein are fluorescent nanoparticles that are particularly useful for imaging tumors in an in vivo or in situ environment. The fluorescent nanoparticles may comprise a gelatin matrix and a fluorophore encapsulated within the gelatin matrix and ionically-bound to the gelatin matrix. The encapsulated fluorophore has an encapsulation concentration selected to provide quenching of fluorophore encapsulated within the gelatin matrix. In contrast, there is a substantial fluorescence for the fluorophore released from the gelatin matrix. In this manner, there is an encapsulated fluorescence value that is substantially less than an unencapsulated fluorescence value, thereby ensuring a good fluorescence tumor to background ratio (TBR), such as TBR that is greater than 2, greater than 3, greater than 5 and greater than 10, including 2≤TBR≤15, and any subranges thereof. The gelatin matrix and fluorophore together form the fluorescent nanoparticle having an effective size that is between 40 nm and 160 nm. Having such a small size nanoparticle that reliably encapsulates fluorophore, can further facilitate uptake of the fluorescent nanoparticles to the tumor microenvironment, while maintaining fluorescent characteristics of the fluorophore during transit from the administration cite (e.g., IV-administration) to the tumor(s) location(s).

The fluorescent nanoparticle more preferably has an effective size that is an average hydrodynamic diameter that is between 50 nm and 90 nm with a standard deviation that is less than or equal to 20 nm.

The gelatin matrix may be a type A gelatin or a type B gelatin, including: the gelatin matrix is a type A (cationic) gelatin and the fluorophore has a net negative charge; or the gelatin matrix is a type B (anioinic) gelatin and the fluorophore has a net positive charge.

The fluorescent nanoparticle may further comprise a material to increase uptake, including by affecting charge and/or charge density. For example, the fluorescent nanoparticle may further comprise diamine molecules covalently conjugated to the gelatin matrix via peptide bond formation to increase an effective positive charge of the gelatin matrix and an increase in cellular uptake.

The fluorescent nanoparticle is compatible with gelatin from acid or base hydrolysis of collagen. For example, the gelatin matrix may be a cationic gelatin configured to encapsulate an anionic fluorophore via a strong electrostatic interaction between the cationic gelatin and the anionic fluorophore.

The fluorophore may be a near-infra-red (NIR) dye. The fluorophore may comprise ICG. The fluorophore may be a NIR dye selected from the group consisting of: indocyanine green (ICG) cyanine dye; Alexa Fluor® (Alexa Fluor 647, 660, 680, 700, 750, 790) fluorescent dyes; Cy® (Cy7 and Cy7.5) (amine-reactive derivative of cyanine); CF® (680, 750, 770, 790, 800) (cyanine-based dye); and IRDye800CW (800 nm channel near-infrared dyes).

The gelatin encapsulated fluorophore is characterized by an encapsulated fluorescence intensity that is less than an unencapsulated (e.g., “free”) fluorescence intensity without a separate quencher molecule in or on the fluorescent nanoparticle. This may be expressed by a ratio of encapsulated to unencapsulated that is greater than a factor of 2, 3 or 5. Similarly, the ratio of tumor to background (a reflection of signal-to-noise) fluorescence is driven, at least in part, by the ratio of encapsulated to unencapsulated, as there is minimal localized gelatin matrix degradation outside the tumor environment.

The fluorophore has an encapsulated fluorescence value when encapsulated within the gelatin matrix and a free fluorescence value (also referred herein as an unencapsulated fluorescence value) when released from the gelatin matrix, wherein the free fluorescence value is greater than the encapsulated fluorescence, including by at least a factor of 2, at least a factor of 3 or at least a factor of 5, at a selected fluorescence emission wavelength and a fluorophore concentration of between 5 μM and 200 μM. The fluorescence value may be intensity at an emission wavelength (such as emission wavelength maximum) or an integrated intensity over an emission wavelength range.

The fluorescent nanoparticle may have a functionalized surface to provide additional control. For example, the fluorescent nanoparticle may further comprise a peptide conjugated to a surface of the fluorescent nanoparticle, wherein the peptide is selected for specific degradation by a cancer cell secreted protease to enhance a tumor selective degradation of the fluorescent nanoparticle to release the encapsulated fluorophore from the gelatin matrix to the desired to-be-imaged tumor and tumor environment. Representative peptides include polypeptides having a dipeptide sequence portion selected from the group consisting of: Phe-Arg, Phe-Lys, Val-Ala, Gly-Leu, and Val-Lys.

Another functionalization may comprise a receptor-specific ligand connected to a surface of the fluorescent nanoparticles, wherein the receptor-specific ligand is selected for a target cell, including a surface-expressed receptor on a cancer cell. Examples include a receptor-specific ligand selected from the group consisting of: folic acid, hyaluronic acid, antibodies and anisamide.

Another functionalization is incorporation of a therapeutic with the fluorescent nanoparticles to provide a one-step visualization and therapy. Examples include for a chemo-photothermal therapy, including with DOX, as described in Chen et al. “Photothermal/matrix metalloproteinase-2 dual-responsive gelatin nanoparticles for breast cancer treatment.” Acta Pharm Sin B. 11(1): 271-282 (2021).

Particularly useful fluorescent nanoparticles include ICG-GNP-II based on type A gelatin, which have a better performance than ICG-GNP-I. ICG-GNP-II allows not only enhanced cellular uptake but also total quenching of encapsulated ICG.

The gelatin matrix of the fluorescent nanoparticle provides a number of important functional benefits. Accordingly, the fluorescent nanoparticle may be described in terms of the gelatin matrix that is configured: to enhance fluorescent particle stability in a biological environment (e.g., the circulatory system for IV-injected fluorescent nanoparticles; the GI system for ingested fluorescent nanoparticles); for in vivo near-infrared (NIR) imaging; to increase an in vivo circulation time of the fluorophore; to facilitate delivery of the fluorescent nanoparticle to and preferentially accumulate in tumors comparted to non-tumor tissue; and/or to enhance fluorescent nanoparticle tumor uptake and gelatin degradation within a tumor environment so that there is minimal fluorescence of the encapsulated fluorophore outside a tumor environment and maximum fluorescence of the fluorophore, including free fluorophore, inside the tumor environment to provide improved tumor detection and contrast.

Also provided are methods of imaging a tumor using any of the fluorescent nanoparticles described herein. The method may comprise the steps of: applying any of the fluorescent nanoparticles to a patient in need of imaging, including tumor-related imaging and waiting a time period for the fluorescent nanoparticles to accumulate and degrade in the tumor. In this manner, at least a portion of the encapsulated fluorophores are released from the gelatin matrix inside the tumor. For the imaging, electromagnetic radiation (light) is applied at an excitation wavelength to excite the fluorophore and an emitted wavelength of electromagnetic radiation generated by the excited fluorophores is measured, wherein the encapsulated fluorophores responsible for the emitted electromagnetic radiation at the emission wavelength have been released from the gelatin matrix to the tumor.

The imaging may comprise near-infra-red imagining for a cancer tumor.

The fluorescent nanoparticles are particularly suited, including the high TBR, for visualization of a tumor boundary for removal of tumors having a negative margin.

The fluorescent nanoparticles are provided to the patient at a dose that is at least 5× less, including 5×-20× less, than the fluorophore that is not encapsulated within the gelatin matrix, such as a dose of 1 mg/kg fluorophore compared to a corresponding conventional dose of 5-10 mg/kg fluorophore not encapsulated within the gelatin matrix, wherein the decreased dose does not adversely impact image quality.

Also provided herein are methods of making any of the disclosed fluorescent nanoparticles. Also provided are methods of enhancing the stability of a fluorophore in a biological environment by encapsulation within a gelatin matrix, including while maximizing a TBR. The method may include adding an acetone solution to an aqueous solution of gelatin; stirring the acetone and aqueous solution of gelatin to precipitate gelatin; collecting the precipitated gelatin; dissolving the collected precipitated gelatin in an acidic (for gelatin type A) or basic solution (for gelatin type B) of DI-water to generate a dissolved gelatin solution; adding acetone to the dissolved gelatin solution under a stirring condition; cross-linking the gelatin in the acetone-dissolved gelatin solution to generate gelatin nanoparticles; collecting the gelatin nanoparticles, wherein the gelatin nanoparticles have an effective density of between 40 nm and 160 nm; and encapsulating a fluorophore with the gelatin nanoparticles, wherein the fluorophore is quenched when positioned within the gelatin nanoparticles

Without wishing to be bound by any particular theory, there may be discussion herein of beliefs or understandings of underlying principles relating to the devices and methods disclosed herein. It is recognized that regardless of the ultimate correctness of any mechanistic explanation or hypothesis, an embodiment of the invention can nonetheless be operative and useful.

### BRIEF DESCRIPTION OF THE DRAWINGS

FIG. 1 is a schematic illustration of a fluorescent nanoparticle administered intravenously with the left panels in the normal vasculature and the right panels in a tumor microenvironment. The relatively more acidic tumor microenvironment results in an increased surface charge on the fluorescent nanoparticle. Upregulated tumor proteolytic activity releases the fluorophore and results in fluorescence activation for the fluorophore preferentially in the tumor microenvironment. The fluorophore contained within the gelatin matrix is quenched with minimal to no fluorescent signal.

FIG. 2 schematically illustrates various optional aspects to provide selective targeting and/or tumor uptake.

FIG. 3 Scanning electron microscopy images of ICG-GNP I of sizes of (A) 80 and (B) 160 nm in diameter. Scale bar=200 nm.

FIG. 4A Absorbance spectra of ICG-free, ICG-GNP I, and ICG-GNP II (equivalent to 10 μM ICG); FIG. 4B Fluorescence intensity of naked ICG (ICG-free), ICG-encapsulating GNP I (ICG-GNP I), and ICG-encapsulating GNP II (ICG-GNP II) at different ICG concentrations, expressed as normalized radiant efficiency

$$( \frac{p / sec / {cm}^{2} / sr}{µ W / {cm}^{2}} ) ;$$

FIG. 4C Release profiles of ICG-GNP I and ICG-GNP II in the presence or absence of trypsin in PBS.

FIG. 5 Cellular uptake of ICG-free, ICG-GNP I and ICG-GNP II (equivalent to 5 μM ICG) by 4T1 cells expressed as radiant efficiency

$$( \frac{p / sec / {cm}^{2} / sr}{µ W / {cm}^{2}} )$$

measured by IVIS. *p<0.05.

FIG. 6A Fluorescence intensity of blood collected from healthy nude mice immediately and 24 hours after administering ICG-free and ICG-GNP I (equivalent to 2 mg/kg ICG). (n=3); FIG. 6B In vivo NIRF imaging of 4T1-tumor bearing mouse after administering ICG-GNP II with a dose equivalent to 1.0 mg/kg ICG. FIG. 6C TBR (tumor to background ratio) for mice administered with ICG-free (2 mg/kg), and ICG-GNP I and ICG-GNP II with a dose equivalent to 1 mg/kg ICG during a 24-hour period, measured by IVIS. Different letters indicate statistically significant differences between groups at each time point. *p<0.05

FIG. 7A Ex vivo NIRF imaging of liver, kidneys, tumor, and muscle tissues after euthanizing the 4T1-tumor bearing mouse at 6-hour post-administration of ICG-GNP II with a dose equivalent to 1.0 mg/kg ICG; FIG. 7B Representative histological and fluorescence images of the tissue specimen showing the contrast between the tumor and normal tissue. The dotted line shows the border between the tumor and normal tissue.

### DETAILED DESCRIPTION

In general, the terms and phrases used herein have their art-recognized meaning, which can be found by reference to standard texts, journal references and contexts known to those skilled in the art. The following definitions are provided to clarify their specific use in the context of the invention.

“Nanoparticle” refers to a particle having a characteristic dimension, such as an effective diameter, that is less than 1000 nm, more preferably between 1 nm and 200 nm, including between 40 nm and 160 nm.

“Encapsulated” refers to a fluorophore that is at least partially surrounded by a material formed from gelatin, such as a gelatin matrix, such that the fluorophore is confined to the gelatin matrix and less accessible to the surrounding environment. In this manner, the effective half-life of the fluorophore is increased relative to a freely administered fluorophore. This is beneficial, particularly for in vivo applications, where the amount or concentration of fluorophore administered to the patient may be reduced while maintaining tumor imaging capability, including good sensitivity (tumor detection) and/or resolution (tumor boundary). Accordingly, encapsulated may be characterized functionally, in terms of an increased half-life, including an at least doubling of half-life, as reflected by a fluorescence emission intensity at a user-selected wavelength that is within at least 20% or 10% of an initial base intensity.

“Encapsulation concentration” refers to the amount of fluorophore encapsulated in the gelatin matrix. Actual concentrations will vary, of course, depending on the fluorophore and/or application of interest. Exemplary concentrations of encapsulated fluorophores in the gelatin matrix include between 1 μM and 50 μM. In general, higher concentration of fluorophores may be used for higher dosing to the patient, with typical fluorophore doses to the patient in the range of 0.1 mg/kg to 50 mg/kg, and any subranges thereof. Another factor, of course, is the total number of fluorescent nanoparticles administered to the patient along with the size of the fluorescent nanoparticles.

“Encapsulated fluorescence value” refers to the fluorescence intensity generated by a fluorophore that is encapsulated by the gelatin matrix. Due to quenching, the encapsulated fluorescence value is low, approaching zero or is zero, corresponding to no detectable fluorescence. The quenching may arise from the concentration of fluorophore in the fluorescent nanoparticle.

“Unencapsulated fluorescence value” refers to the fluorescence intensity generated by a fluorophore that is not encapsulated by the gelatin matrix, such as via proteolytic degradation of the gelatin matrix by a tumor protease, where previously encapsulated fluorophore is effectively unencapsulated and released to the tumor microenvironment. This is also referred herein more generally as “free fluorophore”. The unencapsulated fluorescence value is greater than the encapsulated fluorescence value, such as by between about 2 and 100, including about 2 and 10, and any subranges thereof. Functionally, this provides good TBR, with attendant detection sensitivity and resolution for good tumor boundary imaging useful in tumor resection and for assessment of tumor behavior to any of a variety of cancer treatments. We demonstrate a fluorescent nanoparticle with total quenching at concentrations as low as 20 μM for ICG-GNP I and 10 μM for ICG-GNP II, and minimal fluorescence emission below these thresholds. This is beneficial for enhancing tumor-to-background ratios.

“Substantial fluorescence” refers to an amount of fluorescence that is optically detectable by standard imaging techniques, including for NIR imaging techniques and is optically distinguishable from any fluorescence signal generated by encapsulated fluorophores that are preferably quenched. This semi-quantitative definition reflects that the invention is compatible even if there is low-level fluorescence of the encapsulated fluorophore. The resultant difference in fluorescence is reflected by the TBR values that allows for a straightforward

“Near infrared dye” or NTR dye refers to a fluorophore having absorption in the near infrared area of 700-2000 nm, or between 700 nm to 1400 nm. More generally, the absorption wavelength is just outside the range of visible wavelength by the human eye. Examples include, but are not limited to, Alexa Fluor® (Alexa Fluor 647, 660, 680, 700, 750, 790), Cy® (Cy7 and Cy7.5), CF® (680, 750, 770, 790, 800), and IR© Dyes (IRDye800CW)

“Cellular uptake” refers to a tumor cell that degrades and/or envelops the fluorescent nanoparticle, such that the fluorophore co-locates with a tumor. Uptake can be controlled, such as by varying the charge of the fluorescent nanoparticle and/or functionalizing the surface of the fluorescent nanoparticle.

### Example 1: Overview of Fluorescent Nanoparticles

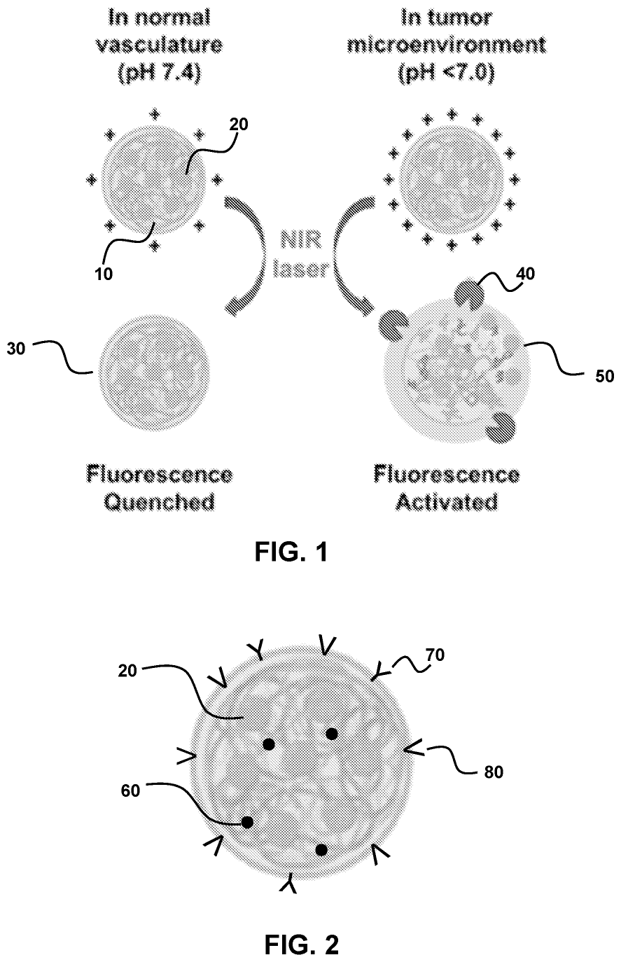

FIGS. 1-2 are schematic illustrations of fluorescent nanoparticles formed from a gelatin matrix 10 with encapsulated fluorophores 20. FIG. 1 shows that in non-tumor tissue, including the normal vasculature having a pH of about 7.4, the fluorophores 20 tend to remain encapsulated (two left panels). In contrast, in the tumor microenvironment, the fluorescence nanoparticles are exposed to tumor proteases and proteinases 40 with a resultant release of fluorophores 20 from gelatin matrix 10 to generate unencapsulated or free fluorophore 50 (two right panels). In this manner, where the encapsulated fluorophore 20 is quenched 30 (e.g., by any number of processes, including energy resonance transfer among the bound fluorophore molecules) for “normal tissue” conditions and freely fluoresces for unencapsulated fluorophores 50. This provides a good TBR, useful for reliably imaging and identifying tumor boundaries.

FIG. 2 illustrates various optional features, such as charged material to impact fluorescent nanoparticle charge, including diamine molecules 60. Also schematically illustrated are a peptide 80 conjugated to fluorescent nanoparticle surface to facilitate tumor selective degradation, and receptor specific ligand 70 to provide targeted delivery to a target cell, including a tumor cell.

### Example 2: Gelatin Matrix

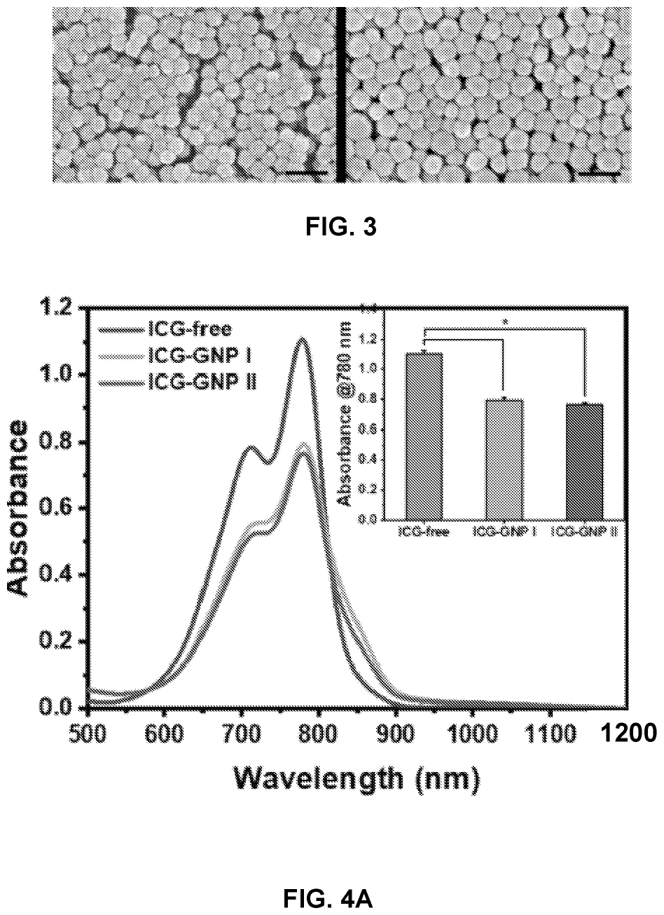

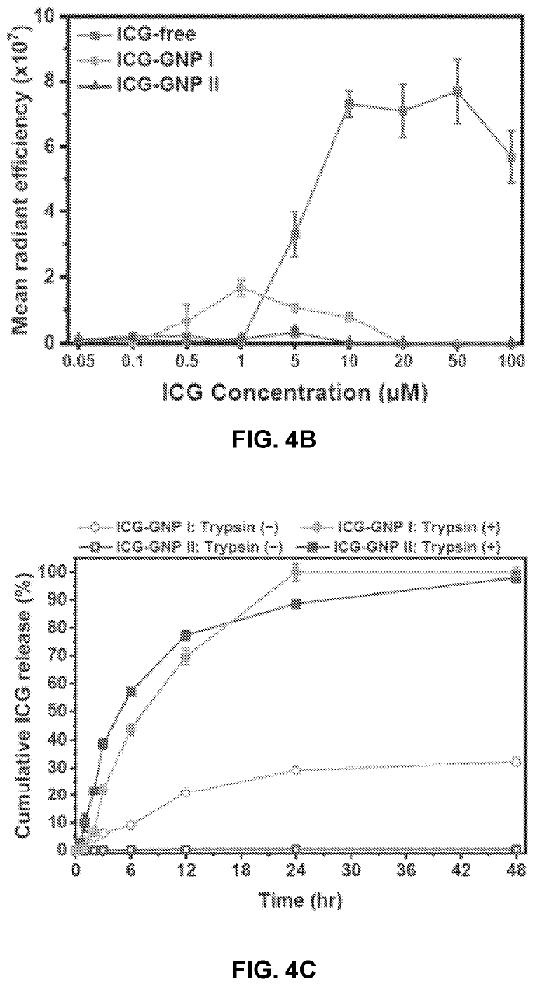

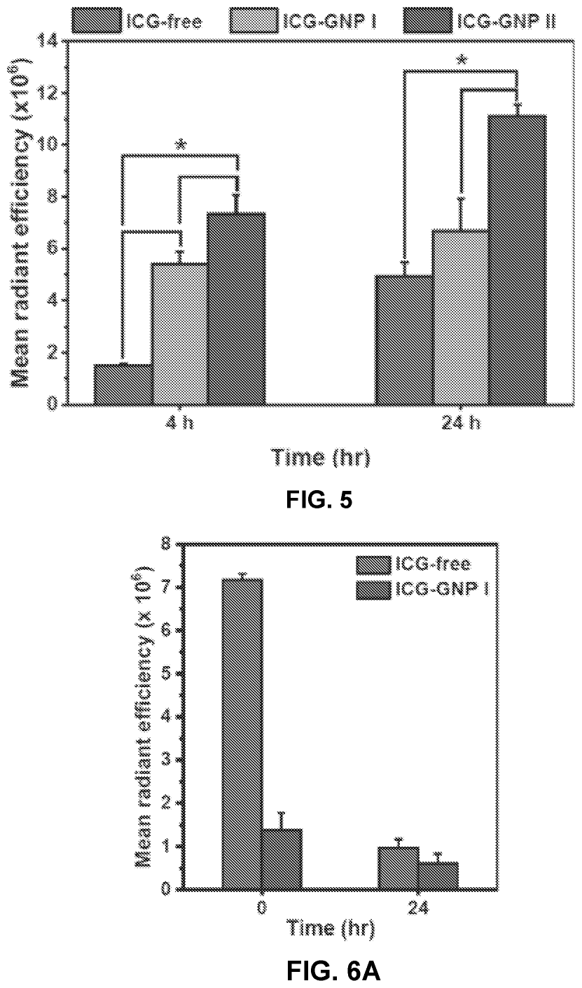

Gelatin, derived from collagen via either acid or base hydrolysis, can carry a net positive or negative charge at physiological pH, respectively. The gelatin nanoparticles (GNPs) formulated from the cationic gelatin have an advantage as a carrier material for anionic molecules, including a NIR fluorophore such as ICG, because of the strong electrostatic interactions of gelatin with the fluorophore to form charge complexes. FIG. 3 shows scanning electron microscopy images of GNPs of two different sizes. The positive charge of the GNPs is increased further by covalent conjugation of diamine molecules to the gelatin via peptide bond formation, enhancing further the electrostatic attraction between the gelatin matrix and the loaded ICG as well as their cellular uptake. Table 1 shows the size uniformity of fluorescent nanoparticles including ICG-GNP I and ICG-GNP II fluorophores, with hydrodynamic diameters of 84 and 76 nm, respectively, and their high loading efficiencies, i.e., 92 and 98%, respectively, for 5 wt % ICG loading. The GNPs exhibits high zeta potentials (ZPs), indicating they are stable in suspension, i.e., 26-28 mV at pH 7, 29-35 mV at pH 6, and 32-39 mV at pH 5. After ICG-loading, these particles still have positive ZPs, i.e., 22-27 mV at pH 7, 24-28 mV at pH 6, and 27-30 mV at pH 5. Such high positive ZP values indicate that the fluorescent nanoparticles are stable, less prone to aggregate, and have a long circulating half-life. The acidic tumor microenvironment may enhance the positive surface charge of fluorescent nanoparticles, facilitating its endocytosis via charge interactions with negatively-charged tumor surfaces. The fluorescent nanoparticles exhibit absorbance spectra with lower intensities compared to free ICG at 10 μM ICG (FIG. 4A). At the primary excitation wavelength for the ICG fluorophores (780 nm), the absorbance of ICG-GNP I and ICG-GNP II is significantly lower than that of ICG-free with no statistically significant difference between the two. The mean fluorescence intensity (MFI) of naked ICG (ICG-free), ICG-GNP I and ICG-GNP II was measured for concentrations equivalent to 0.05 μM-100 μM ICG in PBS using an In Vivo Imaging System (IVIS) and expressed as radiant efficiency (FIG. 4B). All show no fluorescence at concentrations below 0.1 μM. The MFI of ICG-free increases with the concentration to reach a peak at 10 μM, and decreases above 50 μM. In contrast, ICG-GNP I and ICG-GNP II emit minimal fluorescence at concentrations below 20 and 10 μM, respectively, and no fluorescence above these concentrations. The fluorescence quenching shown by the fluorophores encapsulated in gelatin matrix is important for low background fluorescence in healthy tissues, allowing high tumor-to-background ratios (TBR's). The release of fluorophore (e.g., ICG) upon enzymatic degradation of the gelatin matrix results in substantial fluorescence. FIG. 4C shows release profiles measured in the presence or absence of trypsin in PBS. ICG-GNP I exhibit a total ICG release of 9, 21 and 32% in the absence of the enzyme, and 57, 89 and 100% in the presence of the enzyme during the 6-, 12- and 48-hour period, respectively. No release is observed from ICG-GNP II in the absence of trypsin, and 44, 70 and 100% total release in the presence of the enzyme during the 6-, 12- and 48-hour period, respectively. FIG. 5 exhibits the cellular uptake of ICG-free, ICG-GNP I and ICG-GNP II assessed by the IVIS using the 4T1 breast cancer cells. This reveals that ICG-GNP I and ICG-GNP II emit 4 and 5-fold higher fluorescence, respectively, than ICG-free at 4-hour incubation with the cancer cells. The MFI increased for all cases at 24-hour incubation. However, the fluorescent nanoparticles provided herein still emit 1.4-2.3-fold higher fluorescence than ICG-free. These results indicate that the internalization of ICG by the cancer cells is accelerated by the instant fluorescent nanoparticles.

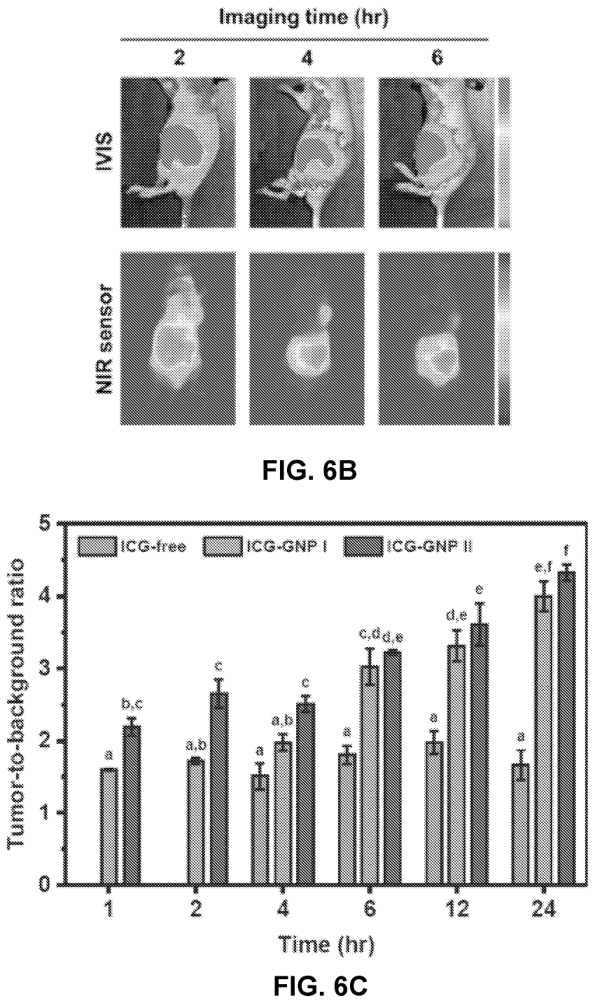

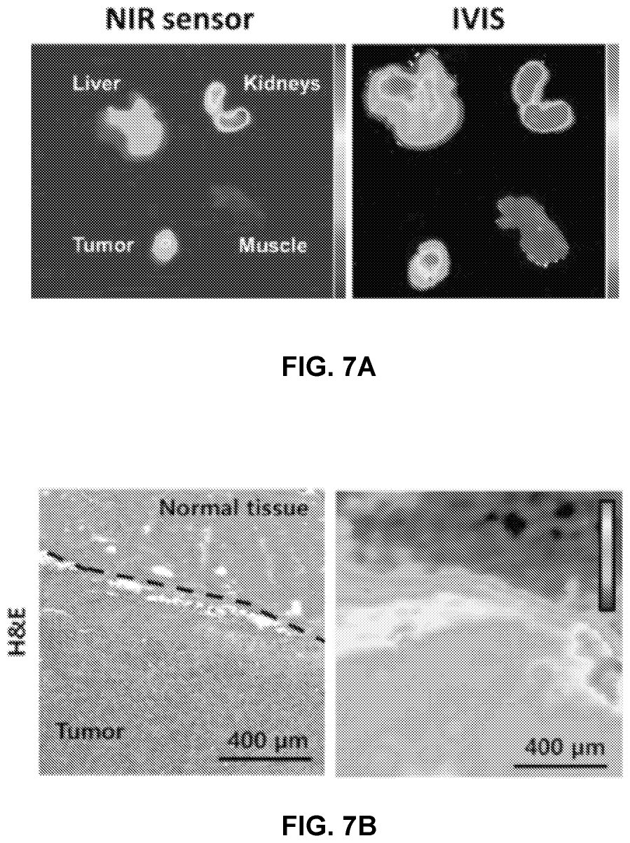

Prior to the in vivo NIR fluorescence imaging, blood samples were collected from healthy nude mice immediately after administering ICG-free and ICG-GNP I (equivalent to 2 mg/kg ICG). FIG. 6A shows significant quenching of ICG emission from ICG-GNP, i.e., >5-fold reduction in MFI, compared to ICG-free (non-encapsulated). This is in line with the in vitro results of FIG. 4B and FIG. 4C, indicating that the fluorescent nanoparticles are intact during systemic circulation in the absence of proteolytic activity and the fluorophore (ICG) emission is quenched. The samples collected at 24-hour post-administration show minimal fluorescence with no significant difference between the two probes, indicating their clearance from the system. FIG. 6B shows in vivo NIR fluorescence (NIRF) images of 4T1-tumor bearing mice administered with ICG-GNP II at a dose equivalent to 1.0 mg/kg ICG during a 6-hour period. Both the IVIS and the NIR sensor exhibit distinctively higher fluorescence intensity in the tumor region compared to the neighboring healthy tissues as early as 4-hour post-administration of ICG-GNP II. The enhanced fluorescence intensity in the tumor region demonstrated by the present invention indicates probe administration on the day of surgery is practical. Considering that ICG doses of 5-10 mg/kg (higher than the FDA-approved limit of 2 mg/kg) have been used for tumor imaging, the accumulation of the fluorescent signal in the tumor displayed by the present invention with the dose equivalent to 1 mg/kg ICG is notable. FIG. 6C shows the TBR's of ICG-free, ICG-GNP I and ICG-GNP II administered into 4T1-tumor bearing mice at the dose of 1 mg/kg ICG except for ICG-free. ICG-free was dosed at 2 mg/kg since it gave no measurable differences in MFI between the tumor and background with the 1 mg/kg dose. However, even with the doubled dose, ICG-free shows low TBRs of ˜1.5 during a 24-hour period. On the other hand, the present invention exhibits a substantially high TBR even at 1-hour post-administration, i.e., 2.2 for ICG-GNP II, which increases to 2.6 at 2-hour. At 6-hour post-administration, ICG-GNP I and ICG-GNP II exhibit TBRs of 3.0 and 3.2, respectively, which increase further with time, reaching 4.0 and 4.3, respectively, at 24-hour. The 4T1-tumor bearing mice were euthanized at 6-hr post-administration of ICG-GNP II to collect the tumor, liver, kidney and muscle tissues for ex vivo NIRF imaging (FIG. 7A). The tumor tissue emits much stronger fluorescence than the muscle, supporting the high TBR of ICG-GNP II at 6-hour post-administration. The strong fluorescence emission from the liver and kidneys indicates the clearance of the particles and their biodegraded components including ICG by these organs.

Visualization of tumor boundaries is crucial in cancer surgery in order to remove tumors with negative margins. FIG. 7B illustrates histological and fluorescence images of a tissue specimen containing both the tumor and normal tissue harvested from a tumor-bearing mouse administered with the fluorescent nanoparticles. The dotted line shows the border between the tumor and normal tissues. The clear contrast between the malignant and normal regions of the specimen indicates that the instant fluorescent nanoparticles facilitate accurate assessment of tumor margins during cancer surgery.

The instant fluorescent nanoparticles achieve TBRs of >2.5, >3.0 and >4.0 at 2, 6 and 24 hour, respectively, with a low dose (1.0 mg/kg ICG). This indicates that the present invention enables efficacious intratumoral ICG delivery to visualize primary tumors for intraoperative NIR imaging.

The effectiveness of the fluorescent nanoparticles is further improved by conjugating the GNP surface with peptides that are specifically degraded by proteases secreted by cancer cells. The fluorescent nanoparticles provided herein are also compatible with active targeting via functionalizing the GNP surface with receptor-specific ligands without chemical modifications of the encapsulated fluorophore. Further, the surface of the GNPs can be conjugated with ligands to actively target corresponding receptors, such as folate and epidermal growth factor (EGFR) receptors.

### Example 3: Nanoparticle Fabrication

Nanoparticle fabrication I: To a 50 ml of 4% (w/v) aqueous solution of gelatin (type A, bloom 300) prepared at 40° C., 50 ml of acetone is added under vigorous stirring, followed by discarding the supernatant and collecting the precipitated gelatin. A designated amount of the purified gelatin is dissolved in DI-water at 40° C. and pH 2.5, followed by the addition of 60 ml of acetone with stirring. When the solution turns turbid, 200 μl of 0.2% glutaraldehyde solution is added dropwise to crosslink the resulting gelatin nanoparticles, GNPs, and stirred for 12 h at room temperature. Subsequently, the GNPs are collected and purified via centrifugation, lyophilized and stored at −20° C. The hydrodynamic diameter of the nanoparticles is measured by dynamic light scattering (Malvern Zetasizer Nano S, Malvern, UK).

Nanoparticle fabrication II: To a 50 ml of 4% (w/v) aqueous solution of gelatin (type A, bloom 300) prepared at 40° C., 50 ml of acetone is added under vigorous stirring, followed by discarding the supernatant and collecting the precipitated gelatin. The purified gelatin is dissolved in DI-H₂O at 40° C. and 5 ml of ethylene diamine is added. After adjusting the pH of the solution to 5.0, 1 g of N-ethyl-N′-(3-dimethylaminopropyl)carbodiimide hydrochloride (EDC) is added and incubated for 18 h at 40° C. with stirring. Afterwards, the solution is dialyzed against DI-water to isolate the aminated gelatin and lyophilized. The amount of primary amine groups in the gelatin is analyzed using 2,4,6-trinitrobenzene sulfonic acid assay. A designated amount of the aminated-gelatin is dissolved in at 40° C. and pH 2.5, 60 ml of acetone is added dropwise with stirring. When the solution turns turbid, 200 μl of 0.2% glutaraldehyde solution is added dropwise to crosslink the resulting GNPs, and stirred for 12 h at 4° C. Subsequently, the GNPs are collected and purified via centrifugation, lyophilized and stored at −20° C. The hydrodynamic diameter of the nanoparticles is measured by dynamic light scattering (Malvern Zetasizer Nano S, Malvern, UK).

Nanoparticle fabrication III: To a 50 ml of 4% (w/v) aqueous solution of gelatin (type A, bloom 300) prepared at 40° C., 50 ml of acetone was added under vigorous stirring, followed by discarding the supernatant and collecting the precipitated gelatin [10, 11]. A designated amount of the purified gelatin was dissolved in DI-water at 40° C. and pH 2.5, followed by the addition of 60 ml of acetone with stirring. When the solution turns turbid, 200 μl of 1% genipin in PBS was added dropwise to crosslink the resulting gelatin nanoparticles, GNPs, and stirred for 24 h at room temperature. Subsequently, the GNPs were collected and purified via centrifugation, lyophilized and stored at −20° C.

Nanoparticle fabrication IV: To a 50 ml of 4% (w/v) aqueous solution of gelatin (type A, bloom 300) prepared at 40° C., 50 ml of acetone was added under vigorous stirring, followed by discarding the supernatant and collecting the precipitated gelatin [10, 11]. A designated amount of the purified gelatin was dissolved in DI-water at 40° C. and pH 2.5, followed by the addition of 60 ml of acetone with stirring. When the solution turns turbid, 2 ml of 10% microbial transglutaminase in PBS was added dropwise to crosslink the resulting gelatin nanoparticles, GNPs, and stirred for 24 h at room temperature. Subsequently, the GNPs were collected and purified via centrifugation, lyophilized and stored at −20° C.

Nanoparticle fabrication V: A designated amount of peptide containing a Phe-Phe-Arg-Asp sequence was added to a mixture of EDC and N-hydroxysuccinimide (NHS) in PBS with stirring. The resulting solution was added to GNP I or GNP II in PBS and reacted for 24 hours at room temperature, followed by centrifugation and washing with dimethylsufoxide (DMSO) to collect peptide-conjugated nanoparticles.

Nanoparticle fabrication VI: A designated amount of folic acid (FA) was mixed with EDC and NHS in 10 ml DMSO under gentle stirring for 30 min. Subsequently, the activated FA solution was added dropwise to a solution of GNP I or GNP II in 10 ml sodium carbonate buffer at pH 10 and reacted for 24 hours at room temperature. The resulting folic acid-conjugated nanoparticles were washed by centrifugation and lyophilized. The amount of conjugated folic acid was measured using the UV-Vis spectrophotometer at 363 nm.

Nanoparticle fabrication VII: A designated amount of GNP I or GNP II in 10 ml sodium carbonate buffer at pH 10 was reacted with a designated amount of 2-iminothiolane for 1 hour at 40° C. under stirring. Subsequently, the activated NeutrAvidin dissolved in sodium carbonate buffer added dropwise to the nanoparticle solution and reacted for 24 hours at 4° C. The resulting NeutrAvidin-conjugated GNP I (Avidin-GNP I) or GNP II (Avidin-GNP II) were washed thoroughly by centrifugation. A designated amount of biotinylated epidermal growth factor (EGF) was dissolved in phosphate buffer saline (PBS, pH 7) and mixed with the Avidin-GNP I or Avidin-GNP II solution for 2 hours at 4° C. The resulting EGF-conjugated GNP I or GNP II were washed by centrifugation.

### ICG Loading, Fluorescence and In Vitro Release

GNP I or GNP II dispersed in 1 ml of ICG in DI-H2O, with a GNP-to-ICG weight ratio of 20, were incubated for 2 hours at 25° C. The resulting suspension was centrifuged (13,000 rpm/5 min) to discard free ICG in the supernatant. The fluorescence intensity of naked ICG (ICG-free), ICG-loaded GNP I (ICG-GNP I) and GNP II (ICG-GNP II) at various ICG concentrations was recorded and quantified using an In Vivo Imaging System (IVIS, Perkin Elmer, Waltham, MA, USA) (ex 740/em 800 nm).

Healthy nude mice were administered with either 100 μl PBS solutions of ICG-free or ICG-GNP I (equivalent to 2 mg/kg ICG) intravenously via retro-orbital injection. 100 μl of blood was collected from the lateral tail vein immediately and 24 hours after the administration, transferred to a 96-well plate, and analyzed their fluorescence intensity using the IVIS (n=3).

ICG-GNP I or ICG-GNP II were dispersed in 1 ml PBS with or without 1% trypsin and incubated in the orbital shaker incubator at 37° C. and 100 rpm. At each time point, the ICG-GNP I or ICG-GNP II suspension was centrifuged to collect 500 μl aliquots from the supernatant. An equal amount of PBS with or without 1% trypsin was added to corresponding suspension before placing the samples back in the incubator. The amount of ICG in each aliquot was determined by measuring the absorbance of ICG at 780 nm using a UV-Vis spectrophotometer (Genesys 10s UV-Vis Spectrophotometer, Thermo Scientific, USA). To assess the amount of ICG loaded to the nanoparticles, ICG-GNP I or ICG-GNP II were incubated in 1 ml PBS with 200 μl 0.25% trypsin at 37° C. for 24 h, followed by centrifugation. The supernatant was collected to measure the amount of ICG by the UV-Vis spectrometer.

Cellular uptake: 4T1 mammary carcinoma cells were seeded in a 24-well plate (106 cells per well) and incubated in RPMI-1640 supplemented with FBS (10% v/v), penicillin (100 IU/ml) and streptomycin (100 g/ml) with 5% CO2 at 37° C. for 24 hours. Subsequently, the cells were incubated with naked ICG (ICG-free), ICG-GNP I or ICG-GNP II (equivalent to 5 μM ICG) at 37° C. for 4 and 24 hours, followed by washing three times with PBS. The resulting cells were subjected to fluorescence measurements by the IVIS (ex 740/em 800 nm) (n=3). Untreated cells were used as a negative control.

Animal tumor model: Female immunodeficient mice (J:NU, 2 months old, average body weight 25 g) were purchased from the Jackson Laboratory (Bar Harbor, Maine, USA). Animal study was performed in strict accordance with the recommendations in the Guide for the Care and Use of Laboratory Animals of the National Institutes of Health. Breast cancer xenograft was established by the orthotopic administration of 100 μl 4T1 cell solution (106 cells per injection) at the mammary fat pad of the second nipple of the left side of each mouse using a 27G½ needle. Anesthesia was maintained by mask inhalation of 1.5-2.0% isoflurane throughout the procedure.

In vivo and ex vivo NIR fluorescence (NIRF) imaging: When the tumor area reached 10 mm² within 14 days, nine tumor-bearing mice were administered with ICG, ICG-GNP I or ICG-GNP II, i.e., three mice for each probe, with a dose equivalent to 1.0 mg/kg ICG and monitored using IVIS. The mice administered with ICG-GNP II were imaged using a bioinspired imaging sensor developed for NIRF image-guided surgery. At each time point, the fluorescence from the primary tumor and surrounding tissues (background) of mice under 2% isoflurane were recorded at 37° C. as regions of interest using the IVIS. A ratio between the mean fluorescence from the tumor and background was assessed and expressed as a TBR. After in vivo imaging, the mice were euthanized to collect the tumors and major organs and muscle tissues subjected to ex vivo imaging. The fluorescence intensity of each organ or tissue was acquired using the IVIS and bioinspired imaging sensor.

Tumor resection and ex vivo imaging: Four tumor-bearing mice administered with ICG-GNP I at a dose equivalent to 1 mg/kg ICG were euthanized at 24 hours to harvest the tumors. The resected tumors were immediately embedded in optimal cutting temperature (OCT) compound and sectioned with a cryostat (CM3050S, Leica, Germany) at −20° C. into a series of 100 μm-thick specimens. The distribution of ICG molecules within each specimen was determined by measuring the fluorescence using a near-infrared fluorescence scanner (Odyssey CLx imager, Li-Cor, Lincoln, NE, USA). Subsequently, each specimen was stained with hematoxylin and eosin (H&E) and imaged with an optical slide scanning system (Nanozoomer slide scanning system, Hamamatsu, Japan).

Statistical analysis: Data are expressed as mean values±SD. Changes in variables were analyzed using one-way ANOVA with Tukey posthoc and paired two-tailed Student's t-tests. Differences between samples were considered as significant at p<0.05 unless otherwise noted.

TABLE 1

| | | | Loading |
|---|---|---|---|
| | Diameterª means ± | ICG-loading^{b} | efficiency^{c} |
| | SD (nm) | (wt %) | (wt %) |
| ICG-GNP I | 83.69 ± 5.22 | 4.61 ± 0.20 | 92.18 ± 3.91 |
| ICG-GNP II | 76.20 ± 9.81 | 4.89 ± 0.07 | 97.78 ± 1.49 |

ªHydrodynamic size;
${b}^{} \frac{( Amount of ICG )}{Amount of ICG - loaded nanoparticles} \times 100 ;$ ${c}^{} \frac{Amount of loaded ICG}{Amount of totl ICG used for loading} \times 100$

### REFERENCES

M. Feng, H. Kang, Z. Yang, B. Luan, and R. Zhou, “Potential disruption of protein-protein interactions by graphene oxide,” J Chem Phys, 144, 225102 (2016) doi: 10.1063/1.4953562.
T. Li, A. Oloyede, and Y. Gu, “Adhesive characteristics of low dimensional carbon nanomaterial on actin,” Appl Phys Lett, 104, 023702 (2014) doi: 10.1063/1.4862200.
Y. Sun, W. Feng, P. Yang, C. Huang, and F. Li, “The biosafety of lanthanide upconversion nanomaterials,” Chem Soc Rev, vol. 44, 1509 (2015) doi: 10.1039/c4cs00175c.
K. S. Soppimath, T. M. Aminabhavi, A. R. Kulkarni, and W. E. Rudzinski, “Biodegradable polymeric nanoparticles as drug delivery devices,” J Control Release, vol. 70, 1 (2001) doi: 10.1016/S0168-3659(00)00339-4.
E. Bilensoy, “Cationic nanoparticles for cancer therapy,” Expert opin drug deliv, 7, 795 (2010) doi: 10.1517/17425247.2010.485983.
D.-H. Ngo et al., “Biological effects of chitosan and its derivatives,” Food Hydrocoll, 51, 200 (2015) doi: 10.1016/j.foodhyd.2015.05.023.
[7] V. Saxena, M. Sadoqi, and J. Shao, “Indocyanine green-loaded biodegradable nanoparticles: preparation, physicochemical characterization and in vitro release,” Int J Pharm, 278, 293 (2004) doi: 10.1016/j.ijpharm.2004.03.032.
[8] M. Fang, J. Yuan, C. Peng, and Y. Li, “Collagen as a double-edged sword in tumor progression,” Tumour Biol, 35, 2871 (2014) doi: 10.1007/s13277-013-1511-7.
[9] J. X. Jiang, J. J. Keating, E. M. Jesus, R. P. Judy, B. Madajewski, O. Venegas, O. T. Okusanya, S. Singhal, Optimization of the enhanced permeability and retention effect for near-infrared imaging of solid tumors with indocyanine green. Am J Nucl Med Mol Imaging, 2015. 5(4): p. 390-400.
[10] E. Joachim, I.-D. Kim, Y. Jin, K. Kim, J.-K. Lee, and H. Choi, “Gelatin nanoparticles enhance the neuroprotective effects of intranasally administered osteopontin in rat ischemic stroke model,” Drug delivery and translational research, vol. 4, no. 5, pp. 395-399, 2014 Dec. 1 2014, doi: 10.1007/s13346-014-0208-9.
[11] I. D. Kim et al., “Robust neuroprotective effects of intranasally delivered iNOS siRNA encapsulated in gelatin nanoparticles in the postischemic brain,” (in eng), Nanomedicine, vol. 12, no. 5, pp. 1219-29, July 2016, doi: 10.1016/j.nano.2016.01.002.
[12]A. F. S. A. Habeeb, “Determination of free amino groups in proteins by trinitrobenzenesulfonic acid,” Analytical Biochemistry, vol. 14, no. 3, pp. 328-336, 1966 Mar. 1 1966, doi: doi.org/10.1016/0003-2697(66)90275-2.
[13]S. Blair et al., “Hexachromatic bioinspired camera for image-guided cancer surgery,” Science Translational Medicine, vol. 13, no. 592, p. eaaw7067, 2021, doi: 10.1126/scitranslmed.aaw7067.

### STATEMENTS REGARDING INCORPORATION BY REFERENCE AND VARIATIONS

All references throughout this application, for example patent documents including issued or granted patents or equivalents; patent application publications; and non-patent literature documents or other source material; are hereby incorporated by reference herein in their entireties, as though individually incorporated by reference, to the extent each reference is at least partially not inconsistent with the disclosure in this application (for example, a reference that is partially inconsistent is incorporated by reference except for the partially inconsistent portion of the reference).

The terms and expressions which have been employed herein are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments, exemplary embodiments and optional features, modification and variation of the concepts herein disclosed may be resorted to by those skilled in the art, and that such modifications and variations are considered to be within the scope of this invention as defined by the appended claims. The specific embodiments provided herein are examples of useful embodiments of the present invention and it will be apparent to one skilled in the art that the present invention may be carried out using a large number of variations of the devices, device components, methods steps set forth in the present description. As will be obvious to one of skill in the art, methods and devices useful for the present methods can include a large number of optional composition and processing elements and steps.

When a group of substituents is disclosed herein, it is understood that all individual members of that group and all subgroups, are disclosed separately. When a Markush group or other grouping is used herein, all individual members of the group and all combinations and subcombinations possible of the group are intended to be individually included in the disclosure.

Every combination of elements described or exemplified herein can be used to practice the invention, unless otherwise stated.

Whenever a range is given in the specification, for example, a size range, a concentration range, a wavelength range or a number range, all intermediate ranges and subranges, as well as all individual values included in the ranges given are intended to be included in the disclosure. It will be understood that any subranges or individual values in a range or subrange that are included in the description herein can be excluded from the claims herein.

All patents and publications mentioned in the specification are indicative of the levels of skill of those skilled in the art to which the invention pertains. References cited herein are incorporated by reference herein in their entirety to indicate the state of the art as of their publication or filing date and it is intended that this information can be employed herein, if needed, to exclude specific embodiments that are in the prior art. For example, when composition of matter are claimed, it should be understood that compounds known and available in the art prior to Applicant's invention, including compounds for which an enabling disclosure is provided in the references cited herein, are not intended to be included in the composition of matter claims herein.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. In each instance herein any of the terms “comprising”, “consisting essentially of” and “consisting of” may be replaced with either of the other two terms. The invention illustratively described herein suitably may be practiced in the absence of any element or elements, limitation or limitations which is not specifically disclosed herein.

The singular terms “a,” “an,” and “the” include plural referents unless context clearly indicates otherwise. Similarly, the word “or” is intended to include “and” unless the context clearly indicates otherwise.

One of ordinary skill in the art will appreciate that materials and methods other than those specifically exemplified can be employed in the practice of the invention without resort to undue experimentation. All art-known functional equivalents, of any such materials and methods are intended to be included in this invention. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

1. A fluorescent nanoparticle for imaging tumors comprising:
a gelatin matrix;
a fluorophore encapsulated within the gelatin matrix and ionically-bound to the gelatin matrix, wherein the encapsulated fluorophore has an encapsulation concentration selected to provide:
quenching of fluorophore encapsulated within the gelatin matrix;
a substantial fluorescence for the fluorophore released from the gelatin matrix; and
wherein the gelatin matrix and fluorophore together form the fluorescent particle having an effective size that is between 40 nm and 160 nm.

2. The fluorescent nanoparticle of claim 1, wherein the effective size is an average hydrodynamic diameter that is between 50 nm and 90 nm with a standard deviation that is less than or equal to 20 nm.

3. The fluorescent nanoparticle of claim 1, wherein the gelatin matrix is a type A gelatin or a type B gelatin.

4. The fluorescent nanoparticle of claim 1, wherein:
the gelatin matrix is a type A (cationic) gelatin and the fluorophore has a net negative charge; or
the gelatin matrix is a type B (anioinic) gelatin and the fluorophore has a net positive charge.

5. The fluorescent nanoparticle of claim 1, further comprising diamine molecules covalently conjugated to the gelatin matrix via peptide bond formation to increase an effective positive charge of the gelatin matrix and an increase in cellular uptake.

6. The fluorescent nanoparticle of claim 1, wherein the gelatin matrix is from acid or base hydrolysis of collagen.

7. The fluorescent nanoparticle of claim 1, wherein the gelatin matrix is a cationic gelatin configured to encapsulate an anionic fluorophore via a strong electrostatic interaction between the cationic gelatin and the anionic fluorophore.

8. The fluorescent nanoparticle of claim 6, wherein the fluorophore comprises ICG.

9. The fluorescent nanoparticle of claim 1, wherein the fluorophore is a near-infra-red (NIR) dye, including an indocyanine green (ICG) cyanine dye.

10. The fluorescent nanoparticle of claim 1, wherein the fluorophore encapsulated with the gelatin matrix has an encapsulated fluorescence intensity that is less than an unencapsulated fluorescence intensity without a separate quencher molecule in or on the fluorescent nanoparticle.

11. The fluorescent nanoparticle of claim 1, wherein the fluorophore has an encapsulated fluorescence value when encapsulated within the gelatin matrix and a free fluorescence value when released from the gelatin matrix, wherein the free fluorescence value is greater than the encapsulated fluorescence value, including by at least a factor of 3, at a selected fluorescence emission wavelength and a fluorophore concentration of between 5 μM and 200 μM.

12. The fluorescent nanoparticle of claim 1, further comprising a peptide conjugated to a surface of the fluorescent nanoparticle, wherein the peptide is selected for specific degradation by a cancer cell secreted protease to enhance a tumor selective degradation of the fluorescent nanoparticle to release the encapsulated fluorophore from the gelatin matrix.

13. The fluorescent nanoparticle of claim 12, wherein the peptide is selected from polypeptides having a dipeptide sequence portion selected from the group consisting of: Phe-Arg, Phe-Lys, Val-Ala, Gly-Leu, and Val-Lys.

14. The fluorescent nanoparticle of claim 1, further comprising a receptor-specific ligand connected to a surface of the fluorescent nanoparticle, wherein the receptor-specific ligand is selected for a target cell, including a surface-expressed receptor on a cancer cell.

15. The fluorescent nanoparticle of claim 14, wherein the receptor-specific ligand is selected from the group consisting of: folic acid, hyaluronic acid, antibodies and anisamide.

16. The fluorescent nanoparticle of claim 1, wherein the gelatin matrix:
enhances fluorescent particle stability in a biological environment;
is configured for in vivo near-infrared (NIR) imaging;
increases an in vivo circulation time of the fluorophore;
is configured to facilitate delivery of the fluorescent nanoparticle to and accumulate in tumors; and/or
is configured to enhance fluorescent nanoparticle tumor uptake and gelatin degradation within a tumor environment so that there is minimal fluorescence of fluorophore outside a tumor environment and maximum fluorescence of the fluorophore inside the tumor environment to provide improved tumor detection and contrast.

17. A method of imaging a tumor, the method comprising the step of:
applying a plurality of the fluorescent nanoparticles of claim 1 to a patient in need of imaging;
waiting a time period for the fluorescent nanoparticles to accumulate and degrade in the tumor, so that at least a portion of the encapsulated fluorophores are released from the gelatin matrix inside the tumor;
applying an excitation wavelength of electromagnetic radiation to excite the fluorophore; and
measuring an emitted wavelength of electromagnetic radiation generated by the excited fluorophores that have been released from the gelatin matrix to the tumor;
thereby imaging the tumor.

18. The method of claim 17, wherein the imaging comprises near-infra-red imagining for a cancer tumor.

19. The method of claim 17, wherein the fluorescent nanoparticles provides visualization of a tumor boundary for removal of tumors having a negative margin.

20. The method of claim 17, wherein the fluorescent nanoparticles are provided to the patient at a dose that is at least 5× less than the fluorophore that is provided as free fluorophore not encapsulated within the gelatin matrix, such as a dose of 1 mg/kg encapsulated fluorophore compared to a corresponding conventional dose of 5-10 mg/kg free fluorophore not encapsulated within the gelatin matrix, wherein the decreased dose does not adversely impact image quality.
